(19) United States
(12) Patent Application Publication
(10) Pub. No.: US 2004/0199040 A1
(43) Pub. Date: Oct. 7, 2004

(54) Process for preparing a microcrystalline wax

(76) Inventors: Arend Hoek, Amsterdam (NL); Hendrik Schadenberg, Amsterdam (NL)

Correspondence Address: Jennifer D Adamson, Shell Oil Company, Intellectual Property, P O Box 2463, Houston, TX, 77252-2463, US

(21) Appl. No.: 10/480,583

(22) PCT Filed: Jun. 13, 2002
(86) PCT No.: PCT/EP02/06584

(30) Foreign Application Priority Data
Jun. 15, 2001 (EP) 01202313.1

(51) Int. Cl. C07C 5/23; C07C 5/25

(52) U.S. Cl. 585/664

(57) ABSTRACT

A process to prepare a microcrystalline wax by contacting under hydroisomerisation conditions a feed, having at least 80 wt % of normal-paraffins and having a congealing point of above 60 ° C., with a catalyst having a noble metal and a porous silica-alumina carrier.

The invention is related to a process for preparing a microcrystalline wax.

It is known to prepare a microcrystalline wax product by means of solvent dewaxing of a petroleum fraction boiling in the base oil range. Examples of such processes are described in The Petroleum Handbook, 6th edition, Elsevier, 1983, Chapter 5 page 265.

It is also known to prepare wax from the product obtained from the Fischer-Tropsch process as for example described in Naidoo P., Watson M. D., Manufacturing and quality aspects of producing hard waxes from natural gas and the resulting HMA performance obtained when using such a wax, 1994 Hot Melt Symposium, TAPPI Proceedings, pages 165-170.

A disadvantage of such a wax based on a Fischer-Tropsch product is that it is too hard to be used in applications as for example in specific hot melt adhesives, as lubricant in PVC manufacturing, chewing gum, petroleum gel, pharmaceutical products, cosmetics, textile impregnation and paper coating applications. The hardness of a wax may be measured by the IP 376 method. Typical PEN values at 43° C. as obtained using this method on commercially available Fischer-Tropsch derived waxes are between 0.2 and 0.6 mm.

It is an object of the present invention to provide a process to prepare a microcrystalline wax having the desired properties, especially having a PEN value (IP 376) at 43° C. of above 0.8 mm

This object is achieved by the following process. Process to prepare a microcrystalline wax by contacting. under hydroisomerisation conditions a feed, comprising at least 80 wt % of normal-paraffins and having a congealing point of above 60° C., with a catalyst comprising a noble metal and a porous silica-alumina carrier.

Preferably the hydroisomerisation conditions are so chosen that preferably less than 10 wt %, and more preferably less than 5 wt %, of the compounds in the feed boiling above 370° C. are converted to products boiling below 370° C. The temperature is suitably between 200 and 400° C. and preferably between 250 and 350° C. The hydrogen partial pressure is suitably between 10 and 100 bar and preferably between 30 and 60 bar. The weight hourly space velocity is suitably between 0.5 and 5 kg/l/h.

The noble metal as present in the catalyst is preferably platinum, palladium or a combination of said metals. The content of noble metal in the catalyst is suitably between 0.1 and 2 wt % and preferably between 0.2 and 1 wt %.

The catalyst carrier may comprise any suitable amorphous silica-alumina. The amorphous silica-alumina preferably contains alumina in an amount in the range of from 2 to 75% by weight, more preferably from 10 to 60% by weight. A very suitable amorphous silica-alumina product for use in preparing the catalyst carrier comprises 45% by weight silica and 55% by weight alumina and is commercially available (ex. Criterion Catalyst Company, USA).

More preferably the amorphous silica-alumina carrier has a certain degree of macroporous pores. The macro-porosity of the carrier is suitably in the range of from 5% vol to 50% vol, wherein the macroporosity is defined as the volume percentage of the pores having a diameter greater than 100 nm. More preferably the carrier has a.! macroporosity of at least 10% vol, even more preferably at least 15% vol and most preferably at least 20% vol. Especially preferred catalysts for use in the process comprise a carrier having a macroporosity of at least 25% vol. Catalysts comprising carriers having a high macroporosity may suffer the disadvantage that the catalyst has a low resistance to damage by crushing. Accordingly, the macroporosity is preferably no greater than 40% vol, more preferably no greater than 38% vol, even more preferably no greater than 35% vol. The side crushing strength of the catalyst is suitably above 75 N/cm, more preferably-above 100 N/cm. The bulk crushing strength of the catalyst is suitably above 0.7 MPa, more preferably above 1 MPa.

References to the total pore volume are to the pore volume determined using the Standard Test Method for Determining Pore Volume Distribution of Catalysts by Mercury Intrusion Porosimetry, ASTM D 4284-88, at a maximum pressure of 4000 bar, assuming a surface tension for mercury of 484 dyne/cm and a contact angle with amorphous silica-alumina of 140°. The total pore volume of the carrier as measured by the above method, is typically in the range of from 0.6 to 1.2 ml/g, preferably in the range of from 0.7 to 1.0 ml/g, more preferably in the range of from 0.8 to 0.95 ml/g.

It will be appreciated that a major portion of the total pore volume is occupied by pores having a pore diameter smaller than 100 nm, that is meso- and micropores. Typically, a major portion of those meso- and micropores has a pore diameter in the range of from 3.75 to 10 nm. Preferably, from 45 to 65% vol of the total pore volume is occupied by pores having a pore diameter in the range of from 3.75 to 10 nm.

In addition to amorphous silica-alumina, the carrier may also comprise one or more binder materials. Suitable binder materials include inorganic oxides. Both amorphous and crystalline binders may be applied. Examples of binder materials comprise silica, alumina, clays, magnesia, titania, zirconia and mixtures thereof. Silica and alumina are preferred binders, with alumina being especially preferred. The binder, if incorporated in the catalyst, is preferably present in an amount of from 5 to 50% by weight, more preferably from 15 to 40% by weight, on the basis of total weight of the carrier. Catalysts comprising a carrier without a binder are preferred for use in the process of this invention. The above preferred catalyst can be obtained by the process as for example described in EP-A-666894. Further examples of suitable catalysts are described in WO-A-200014179, EP-A-532118, EP-A-587246, EP-A-532116, EP-A-537815 and EP-A-776959.

The feed comprises at least 80 wt %, and preferably at least 85 wt %, of normal-paraffins. The feed has a congealing point of above 60° C. and preferably above 90° C. and even more preferably above 95° C. The upper limit for the melting temperature and congealing point is suitably below 125° C. The PEN value as determined by IP 376 at 43° C. is preferably smaller than 0.7 mm. The oil content as determined by ASTM D 721 will typically be low, for example smaller than 1 wt % and more typically less than 0.5 wt %. The kinematic viscosity at 150° C. of the feed is preferably above 7 cSt. The feed suitably contains less than 0.1 ppm sulphur in order not to deactivate the catalyst.

Such a preferred feed is suitably obtained in a Fischer-Tropsch synthesis. Such a process can prepare fractions having a high content of normal paraffins. Examples of such processes are the so-called commercial Sasol process, the commercial Shell Middle Distillate Process or by the non-commercial Exxon process. These and other processes are for example described in more detail in EP-A-776959, EP-A-668342, U.S. Pat. No. 4,943,672, U.S. Pat. No. 5059299, WO-A-9920720. A preferred Fischer-Tropsch process to prepare the feed for the present process is described in WO-A-9934917. This process is preferred because it yields a Fischer-Tropsch product, comprising a sufficient amount of the fraction having a congealing point of higher than 60° C. and higher.

Examples of commercially available Fischer-Tropsch derived wax products which can be used as feedstock are SX100 as described in “The Markets for Shell Middle Distillate Synthesis Products”, Presentation of Peter J. A. Tijm, Shell International Gas Ltd., Alternative Energy '95, Vancouver, Canada, May 2-4, 1995 and Paraflint H1 as marketed by Schumann Sasol Ltd (SA).

The synthesis product as directly obtained in the Fischer-Tropsch process is preferably hydrogenated in order to remove any oxygenates and saturate any olefinic compounds present in such a product. Such a hydrotreatment is described in for example EP-B-668342. The feed for the present product can be obtained by separating the lower boiling compounds and optionally higher boiling compounds from the Fischer-Tropsch product by means of distillation or any other suitably separation technique.

The microcrystalline wax as obtained by the present process, optionally after a de-oiling step, may find application in the earlier mentioned applications. The wax may be used as a lubricant for processing of PVC (poly vinyl chloride), for example for rigid PVC extrusion. The wax may also be used as a carrier wax for polytheylene master batches. Furthermore it has been found that the wax product has a better compatible with polar compounds as compared to the feed. For example the wax product is better compatible with polar pigments.

The invention is also directed to the soft microcrystalline wax as such which is believed to be a novel wax having the following properties. Fischer-Tropsch derived wax having a congealing point as determined by ASTM D 938 of between 85 and 120 and more preferably between 95 and 120° C. and a PEN at 43° C. as determined by IP 376 of more than 0.8 mm and preferably more than 1 mm. The wax is further characterized in that it preferably comprises less than 1 wt % aromatic compounds and less than 10 wt % naphthenic compounds, more preferably less than 5 wt % naphthenic compounds. The mol percentage of branched paraffins in the wax is preferably above 33 and more preferably above 45 and below 80 mol% as determined by C₁₃ NMR. This method determines an average molecular weight for the wax and subsequently determines the mol percentage of molecules having a methyl branch, the mobl percentage of molecules having an ethyl branch, the mol percentage of molecules having a C₃ branch and the mol percentage having a C₄⁺ branch, under the assumption that each molecule does not have more than one branch. The mol % of branched paraffins is the total of these individual percentages. This method calculated the mol% in the wax of an average molecule having only one branch. In reality paraffin molecules having more than one branch may be present. Thus the content of branched paraffins determined by different method may result in a different value.

The oil content as determined by ASTM D 721 is typically below 2 wt %. The lower limit is not critical. Values of above 0.5 wt % may be expected, but lower values can be achieved depending on the method in which the wax is obtained. Most likely the oil content will be between 1 and 2 wt %. The kinematic viscosity at 150° C. of the wax is preferably higher than 8 cSt and more preferably higher than 12 and lower than 18 cSt.

The invention will now be illustrated with the following non-limiting examples.

### EXAMPLE 1

A wax fraction as obtained from the Fischer-Tropsch synthesis product as obtained in Example VII using the catalyst of Example III of WO-A-9934917 was continuously fed to a hydroisomerisation step. The properties of the feed are described in Table 1.

In the hydroisomerisation step the fraction was contacted with a hydroisomerisation catalyst of Example 1 of EP-A-532118. The hydroisomerisation step was performed at 30 bara and at a temperature of 325° C. The remaining conditions were so chosen that the conversion of the feed to products boiling below 370° C. was below 10 wt %.

The product as obtained in the hydroisomerisation were analysed and the results are presented in Table 1.

TABLE 1

| | SX100* | Paraflint H1** | feed | Product |
|---|---|---|---|---|
| Congealing point (ASTM D | 97.3 | 100 | 104.5 | 101.0 |
| 938; ° C.) | | | | |
| Drop melting point (ASTM | 110.0 | 113.5 | 116.7 | 113.4 |
| D 127) (° C.) | | | | |
| PEN at 25° C. (IP 376) (mm) | 0.1 | 0.1 | 0.1 | 0.8 |
| PEN at 43° C. | 0.4 | 0.4 | 0.2 | 1.6 |
| PEN at 65° C. | 1.1 | 1.7 | 0.7 | 4.0 |
| Oil content (ASTM D 721; | <0.1 | Not measured | <0.1 | 1.6 |
| wt %) | | | | |
| Kinematic viscosity at | 7.97 | Not measured | 14 | 13.8 |
| 150° C. (ASTM D 445) | | | | |
| Crystal structure by | Yes | Yes | Yes | Yes |
| microscopic observation | | | | |
| % branching (mol %) | 9 | 11.5 | 11.1 | 60*** |

*SX100 is a Fischer-Tropsch wax as marketed by Shell Malaysia bhp
**Paraflint H1 is a Fischer-Tropsch derived wax marketed by Schumann Sasol
***36 mol % mono-methyl branched paraffin molecules, 8 mol % mono-ethyl branched paraffin molecules, 4 mol % mono-propyl branched paraffin molecules and 12 C₄⁺ mono-branched paraffin molecules.

1. A process for preparing a microcrystalline wax comprising:
contacting a feed, comprising compounds comprising at least 80 wt % of normal-paraffins and having a congealing point of above 60° C., with a catalyst comprising a noble metal and a porous silica-alumina carrier wherein the process is performed under hydroisomerization conditions.

2. The process of claim 1, wherein less than 10 wt % of the compounds in the feed boiling above 370° C. are converted to products boiling below 370° C.

3. The process of claim 2, wherein less than 5 wt % of the compounds in the feed boiling above 370° C. are converted to products boiling below 370° C.

4. The process of claims 1-3, wherein the hydroisomerisation conditions comprise a temperature of between 250° C. and 350° C., a hydrogen partial pressure of between 30 bar and 60 bar and a weight hourly space velocity of between 0.5 kgl/h and 5 kg/l/h.

5. The process of claims 1-4, wherein the noble metal is selected from the group consisting of platinum, palladium or a combination thereof.

6. The process of claims 1-5, wherein the amorphous silica-alumina carrier has a macroporosity in the range of from 5% vol to 50% vol, wherein the macroporosity is defined as the volume percentage of the pores having a diameter greater than 100 nm.

7. The process of claim 6, wherein the macroporosity is between 10 vol % and 40 vol %.

8. The processof claims 1-7, wherein the amorphous silica-alumina carrier comprises alumina in an amount in the range of from 2% to 75% by weight.

9. The process of claim 8, wherein the alumina content is between 10% to 60% by weight.

10. The process of claims 1-9, wherein the total pore volume of the carrier is in the range of from 0.6 ml/g to 1.2 ml/g.

11. The process of claims 1-10, wherein the feed is obtained by means of a Fischer-Tropsch synthesis.

12. The process of claims 1-11, wherein the feed has a congealing point of between 95° C. and 120° C.

13. The process of claims 1-12, wherein the PEN at 43° C. of the feed as determined by IP 376 is smaller than 0.7 mm.

14. The process of claims 12-13, wherein the microcrystalline wax as obtained has a congealing point of between 95-120° C. and a PEN at 43° C. as determined by IP 376 of more than 0.8 mm.

15. The process of claim 14, wherein the PEN at 43° C. is more than 1.0 mm.

16. A composition comprising a microcrystalline wax having a congealing point of between 95° C. and 120° C. and a PEN at 43° C. as determined by IP 376 of more than 0.8 mm.

17. The wax of claim 16, wherein the PEN at 43° C. as determined by IP 376 is greater than 1.0 mm.

18. The wax of of claims 16-17, wherein the content of branched paraffins is greater than 33 wt %.

19. The wax of claims 16-18, wherein the content of aromatic compounds is less than 1 wt % and the content of naphthenics compounds is less than 10 wt %.

20. The wax of claims 16-19, wherein the oil content as determined by ASTM D 721 is below 2 wt %.
